(19) United States
(12) Patent Application Publication
(10) Pub. No.: US 2023/0218448 A1
(43) Pub. Date: Jul. 13, 2023

(54) ABSORBENT INTIMATE HYGIENE PRODUCT FOR PREVENTING AND TREATING INFECTION

(71) Applicant: Tünde RATKÓ, Szentendre (HU)

(72) Inventor: Tünde RATKÓ, Szentendre (HU)

(21) Appl. No.: 18/009,809

(22) PCT Filed: Jun. 14, 2021
(86) PCT No.: PCT/HU2021/050040

§ 371 Date: Dec. 12, 2022

(30) Foreign Application Priority Data
Jun. 12, 2020 (HU) U2000106
Jun. 12, 2020 (WO) PCT/HU2020/050025
Jun. 11, 2021 (HU) P21 00228

(51) Int. Cl. A61F 13/20; A61F 13/34; A61L 15/18

(52) U.S. Cl. CPC A61F 13/2074; A61F 13/202; A61F 13/2034; A61F 13/34; A61L 15/18; A61F 2013/8414

(57) ABSTRACT

Devices according to the invention use a metal-based material having antimicrobial, biocidal, bactericidal, virucidal and/or fungicidal effect. Metals having antimicrobial properties can be toxic to microorganisms of interest (microbes), such as bacteria and/or fungi.

### FIELD OF THE INVENTION

The invention relates to an absorbent intimate sanitary/hygiene product that comprises an absorbent fibrous unit and has an intimate body surface contacting side, and at least a part of said intimate body surface contacting side is provided with an antimicrobial, metal-based fiber.

### TECHNICAL BACKGROUND

Vaginal tampons and sanitary napkins are feminine hygiene products that are made up of strong absorbent fibers to collect certain liquid. Baby diapers and adult diapers are hygienic products that are also made up of strong absorbent fibers to collect certain liquid.

WO2008024426 discloses nanosilver-containing absorbent articles, such as tampons, and sanitary napkins. The tampon of the invention disclosed in the document, the nanosilver particles are provided to an absorbent body arranged in the inside of the tampon and dissolve from there, thereby reducing the development of possible infections.

US2013237933A1 discloses a tampon structure comprising at least one absorbent carbonaceous base with at least two precious metals, an absorber, a surface layer and a rope. In an aqueous medium, particles of precious metals may dissolve to maintain vaginal health. The precious metals used may be the following metals known for their antimicrobial activity: silver, gold, palladium, platinum, copper, aluminum, nickel, cobalt, zinc, silicon, calcium, titanium, chromium and combinations thereof.

In addition to their absorbent properties, the above-described tampons are also provided with an antimicrobial metal. The absorbent body swells under the influence of moisture so that metal particles dissolve from the antimicrobial metal, such as metal ions, metal atoms, nanoparticles, which have toxic effects for certain microorganisms.

After insertion, a tampon with antimicrobial metal-based substance(s) in contact with moisture is suitable for preventing possible infections which develop in the vagina such as toxic shock syndrome (TSS), bacterial vaginosis (BV), vaginal yeast infections, and for preventing cervical lesions, wounds, uterin wounds, epithelial lesions due to the infections. Optionally, the tampon that is applied may be suitable for forming an anti-biofilm on the epithelial wound to isolate and protect the wound from external irritants, thereby promoting the process of scar formation and wound healing. Furthermore, the antimicrobial materials may be suitable for both the treatment of pre-existing infections, primarily in combination therapy, and for post-infection treatment.

The tampons described above contain the antimicrobial metal or metals inside the absorbent, fibrous body of the tampon. In these solutions, the antimicrobial material is arranged inside the tampon body, thus the amount of dissolved metal particles depends on the amount of liquid absorbed by the tampon. With a smaller amount of liquid, the amount of dissolved metal particles may be so small that they are not suitable for developing an effective antimicrobial effect.

Therefore the antimicrobial materials of these vaginal tampons can be less effective both in promoting the healing of epithelial wounds and in preventing, treating and/or providing aftercare the vaginal infections.

CN201505224U, a Chinese utility model, discloses a multilayer antibacterial and sanitary napkin, all layers are fixedly connected together; wherein the surface layer consists of an antibacterial layer comprising nanosilver. The nanosilver antibacterial layer is made by soaking in cotton or other fabric material.

This product does not contain antimicrobial fibers and its production is a complex process.

The sanitary products of the present invention contain antimicrobial fibers that are easily accessible to body moisture, preferably in an exposed area, such as on or near the surface of the body, or in an area that facilitates insertion or withdrawal (in a preferred embodiment the tampon withdrawal fiber is antimicrobial), so that even a small amount of absorbed liquid can provide an adequate and effective amount of dissolved metal particles.

### BRIEF DESCRIPTION OF THE INVENTION

The present invention relates to an absorbent intimate sanitary/hygiene product that is provided with a metal-based fiber having antimicrobial activity or designed to receive such a fiber, and further comprises an absorbent fibrous unit and has an intimate body surface contacting side, wherein the tampon, when impregnated with a liquid, preferably a body fluid, releases metal particles from the antimicrobial metal-based fiber through its surface in contact with the intimate body surface.

Preferably, the antimicrobial metal-based fiber is arranged near, substantially parallel to, the surface in contact with the intimate body surface.

The present invention relates to an absorbent intimate hygiene product that comprises an absorbent fibrous unit and has an intimate body surface-contacting side, wherein at least a part of the intimate body surface-contacting surface is provided with an antimicrobial metal-based fiber or designed to receive such a fiber.

The present invention relates to an absorbent intimate hygiene product that comprises an absorbent fibrous unit and has an intimate body surface-contacting side, and is provided with an antimicrobial metal-based fiber, wherein the antimicrobial metal-based fiber is near to the intimate body surface-contacting surface, at a distance of not more than 30%, 25% or 20%, preferably not more than 15%, 10% or 5%, in proportion to the total thickness of the product, preferably in the case of an ellipsoidal cross-section.

Preferably, at least a part of intimate body surface-contacting surface of the product is provided with an antimicrobial metal-based fiber and the fiber is arranged at a distance of not more than 30%, 25% or 20%, preferably not more than 15%, 10% or 5% of the surface, in proportion to the total thickness of the product, and is preferably substantially parallel to the surface.

In an embodiment, the fiber is arranged on the outer side of the surface.

In another embodiment, the fiber is arranged on the inner side of the surface.

Preferably the fiber is one or more fibers, preferably more fibers.

In a preferred embodiment, in addition to the one or more fibers near the surface, the product contains antimicrobial fibers only in an amount of less than the one or more fibers, preferably only one such fiber in addition to the one or more fibers near the surface or contains no such fiber.

In a preferred embodiment, the invention relates to a vaginal tampon.

An embodiment of the tampon comprises an absorbent fibrous unit and is either provided with a metal-based fiber or is designed to receive such a fiber, wherein the metal-based fiber is the withdrawal fiber.

In a preferred embodiment of the tampon, the tampon (or tampon body) is provided with an antimicrobial metal-based fiber, wherein the antimicrobial metal-based fiber is arranged near the intimate body surface contacting surface at a distance of not more than 30%, 25% or 20%, preferably not more than 15%, 10% or 5% of the total thickness, i.e., diameter, of the tampon.

In a preferred embodiment of the tampon, at least a part of the intimate body surface contacting surface of the tampon body is provided with an antimicrobial metal-based fiber or is designed to receive such a fiber.

Preferably, the antimicrobial metal-based fiber is arranged near, substantially parallel to, the surface in contact with the intimate body surface.

In a preferred embodiment of the tampon, the tampon comprises an inner part having a metal-based antimicrobial fiber arranged thereon in such a way that the fiber is arranged on at least a part of the surface of the inner part, wherein the tampon further comprises an outer part that surrounds the inner part of the tampon body, the thickness of which is not more than 30%, 25% or 20%, preferably not more than 15%, 10% or 5% of the total thickness of the tampon, i.e. the diameter of the tampon.

In a preferred embodiment, the antimicrobial fiber may be embedded in a groove formed in the inner part of the tampon or may be attached to the surface of the inner part. In a preferred embodiment, the antimicrobial fiber may be embedded in a groove formed in the surface of the tampon body or may be attached to the surface of the tampon body.

In a preferred embodiment, the tampon comprises a top layer which may be any fabric used in a hygiene product. In a preferred embodiment, the top layer may be comprised of a functional material, such as a breathable textile, a probiotic fabric, an antimicrobial fabric, etc. Alternatively, the antimicrobial fiber is arranged on the surface of the inner part, and all or part of the surface of the top layer is permeable to, optionally in a specific pattern, for metal particles releasing the interior part of the tampon.

The tampon is preferably a single-use, disposable tampon.

In a preferred embodiment, the intimate hygiene product is a vaginal tampon 10, wherein the tampon 10 comprises:
a cylindrical tampon body 20 having an insertion end 22 and a withdrawal end 23, wherein the tampon body 20 comprises a absorbent, fibrous unit 60 pressed into a self-supporting shape;
a withdrawal fiber 30 connected to the tampon body 20 near its withdrawal end 23;
   wherein the tampon body 20 comprises at least one groove 40 along the outer surface of the tampon body 20, characterized in that the at least one groove 40 has a depth that is adapted to embed at least one antimicrobial fiber 50, wherein the at least one antimicrobial fiber 50 is arranged in at least one section of the at least one groove 40.

In a preferred embodiment, the withdrawal fiber 30 also acts as an antimicrobial fiber 50.

Preferably, the at least one antimicrobial fiber 50 in the vaginal tampon 10 is a metal-based fiber containing metal particles 51 toxic to microorganisms of interest, selected in particular from the following materials: silver, copper, zinc, gold, palladium, platinum, aluminum, nickel, cobalt, silicon, calcium, titanium, chromium, molybdenum, vanadium, tungsten, gallium, selenium, tin, tellurium and/or a combination thereof.

In another preferred embodiment, at least the antimicrobial fiber 50 in the vaginal tampon 10 is a fiber containing metal particles that comprises a non-metallic carrier and metal particles in or on the surface thereof, wherein the metal particles are preferably metal ions, metal atoms or nanoparticles, i.e. the metal is present in colloidal form. Preferably, the non-metallic carrier fiber is impregnated with a solution or suspension of the metal particles. The non-metallic carrier is preferably a fiber made of a biologically tolerable, absorbent material compatible with the use of the tampon, preferably a fiber of plant origin, e.g. bombast, cotton, or synthetic fiber, e.g. polyester, or fiber of animal origin e.g. wool. Preferably, the metal is selected from the following materials: silver, copper, zinc, gold, palladium, platinum, aluminum, nickel, cobalt, silicon, calcium, titanium, chromium, molybdenum, vanadium, tungsten, gallium, selenium, tin, tellurium and/or a combination thereof.

Preferably, in the vaginal tampon 10 of the present invention, the metal particles 51 have biocidal, bactericidal, virucidal, fungicidal effect and/or a combination thereof.

Preferably, the diameter of the at least one antimicrobial fiber 50 in the vaginal tampon 10 of the present invention is at least about 0.01 mm, preferably at least 0.05 mm, preferably between 0.1 mm and 4 mm, preferably between 0.1 mm and 3 mm or between 0.5 mm and 4 mm or between 0.5 mm and 3 mm, more preferably between 0.5 mm and 2 mm, most preferably between 0.5 mm and 1 mm.

Preferably, in the vaginal tampon 10 of the present invention, the at least one groove 40 is formed in a continuous, intermittent, spiral, zigzag or wavy shape.

Preferably, in the vaginal tampon 10 of the present invention, the at least one groove 40 has a depth that is adapted to embed a plurality of antimicrobial fibers 50, wherein the material of the antimicrobial fibers 50 varies.

Preferably, in the vaginal tampon 10 of the present invention, the at least one groove 40 has a depth that is adapted to embed a plurality of antimicrobial fibers 50, wherein the material of the antimicrobial fibers 50 is the same.

Preferably, in the vaginal tampon 10 of the present invention, the at least one antimicrobial fiber 50 covers the outer surface of the tampon body 20 near the insertion end a larger portion 22 than near the withdrawal end 23 of the tampon body 20.

Preferably, in the vaginal tampon 10 of the present invention, the at least one antimicrobial fiber 50 together with the withdrawal fiber 30 is guided near the withdrawal end 23 of the tampon body 20.

Preferably, in the vaginal tampon 10 of the present invention, the cylindrical tampon body 20 has a length of about 3 to 15 cm, preferably 3 to 11 cm, and a diameter of about 0.5 cm to 1.5 cm in the dry state and a diameter of about 1.5 cm to 4 cm, preferably about 2.5 cm in the wet state.

Preferably, the vaginal tampon 10 according to the invention is provided with an applicator.

Preferably, in the vaginal tampon 10 of the present invention, the outer surface of the tampon body 20 is provided with a top sheet 70 which is permeable to the metal particles 51.

Preferably, in the vaginal tampon 10 of the present invention, the top sheet 70 is a perforated fabric of breathable material that is additionally provided with a probiotic and/or antimicrobial material.

Preferably, in the vaginal tampon 10 of the present invention, the tampon body 20 comprises at least one antimicrobial fiber 50 disposed on at least a portion of the length of the tampon body 20 and arranged inside the absorbent fibrous unit 60.

Preferably, in the vaginal tampon 10 of the present invention, the tampon body 20 contains antimicrobial material(s) inside the absorbent fibrous unit 60.

The invention further relates to a tampon kit comprising a vaginal tampon 10 according to the invention and a top sheet 70a comprising or consisting of an antimicrobial metal, wherein the top sheet 70a covers at least a part of the outer surface of the tampon body 20.

Preferably, in the tampon kit of the present invention, the top sheet 70a is a foldable and/or cuttable top sheet.

Preferably, the tampon kit of the present invention comprises vaginal tampons 10 of different dimensions and top sheets 70a of different dimensions covering at least a part of the outer surface of the tampon 10 of a specific size.

Preferably, the tampon kit of the present invention comprises a plurality of vaginal tampons 10 according to the invention, a top sheet 70a comprising or consisting of an antimicrobial metal and a top sheet 70b having a probiotic effect.

Preferably, in the tampon kit of the present invention, the top sheets 70a, 70b are foldable and/or cuttable top sheets.

Preferably, the tampon kit of the present invention comprises tampons 10 of different sizes.

Preferably, the tampon kit of the present invention comprises vaginal tampons 10 having different dimensions and top sheets 70a, 70b having different dimensions covering at least a part of the outer surface of the tampon 10 of a specific size.

Preferably, in the tampon kit of the present invention, the antimicrobial and probiotic top sheets 70a, 70b have different patterns, structures, colors and/or packaging.

In another preferred embodiment, the invention further relates to a sanitary napkin comprising
an absorbent pad body having
an inner side in contact with the user's skin surface, and
an outer side, and
in the pad body, a flexible absorbent fibrous unit is disposed between its inner and outer sides,
   characterized in that the pad body comprises an antimicrobial metal-based fiber, wherein at least a part of the inner surface in contact with the user's skin surface is provided with an antimicrobial metal-based fiber, and/or

the antimicrobial metal-based fiber is arranged near the surface in contact with the skin surface, at a distance of not more than 30%, 25% or 20%, preferably not more than 15%, 10% or 5% of the total thickness of the product, preferably in case of an ellipsoid cross-section of its diameter, and/or

preferably, the antimicrobial metal-based fiber is arranged near, substantially parallel to, the surface in contact with the intimate body surface.

In another preferred embodiment, the invention further relates to a sanitary napkin comprising
an absorbent pad body having
an inner side in contact with the user's skin surface (surface in contact with an intimate body surface), and
an outer side, and
in the pad body, a flexible absorbent fibrous unit is disposed between its inner and outer sides,
   characterized in that at least a part of the inner surface of the pad body that is in contact with the user's skin surface is provided with an antimicrobial metal-based fiber or metal particles.

Preferably, the antimicrobial metal-based fiber or metal particle has a biocidal, virucidal, bactericidal and/or fungicidal effect or a combination thereof.

Preferably, the antimicrobial metal-based fiber is arranged near, substantially parallel to, the surface in contact with the intimate body surface.

Preferably, in the sanitary napkin of the present invention, the antimicrobial metal-based fiber is in the form of a spiral, wavy or zigzag shape.

Preferably, the sanitary napkin of the present invention is the pad shown in FIG. 11a (B1a) or 11b (B1b).

Preferably, the invention relates to a sanitary napkin comprising an absorbent pad body having
an inner side in contact with the user's skin surface, and
an outer side, and
a flexible, absorbent fibrous unit between its inner and outer sides, wherein
   one side and a part of the fibrous unit form a first part, and
   the other side and the other part of the fibrous unit form a second part,
   wherein the first part is provided at least partly with an antimicrobial metal-based fiber, and the second part is made of a fabric material which is permeable to metal particles, e.g. for metal ions, metal atoms or nanoparticles derived from the fibers arranged in its structure.

In a preferred embodiment, the antimicrobial metal-based fiber in the sanitary napkin of the present invention is a metal fiber.

In another preferred embodiment, the antimicrobial metal-based fiber in the sanitary napkin of the present invention is a fiber containing metal particles that comprises a non-metallic carrier and metal particles in or on the surface thereof, wherein the metal particles are preferably metal ions, metal atoms or nanoparticles, i.e. the metal is present in colloidal form. Preferably, the non-metallic carrier fiber is impregnated with a solution or suspension of the metal particles. The non-metallic carrier is preferably a fiber made of a biologically tolerable, absorbent material compatible with the use of the sanitary napkin, preferably of plant origin e.g. bombast, cotton, or synthetic fiber e.g. polyester, or fiber of animal origin e.g. wool. Preferably, the metal is selected from the following materials: silver, copper, zinc, gold, palladium, platinum, aluminum, nickel, cobalt, silicon, calcium, titanium, chromium, molybdenum, vanadium, tungsten, gallium, selenium, tin, tellurium and/or a combination thereof.

Preferably, in the sanitary napkin of the present invention,

the first part is arranged on the inner side and they are formed as a unitary part, and the second part is arranged on the outer side and they are formed as a unitary part.

Preferably, in the sanitary napkin of the present invention, the antibacterial metal-based fiber is in the form of a spiral, wavy or zigzag shape.

Preferably, the sanitary napkin of the present invention is the pad shown in FIG. 12a (B2a) or 12b (B2b).

Preferably, in the sanitary napkin of the present invention, the antimicrobial material is inserted into the absorbent fiber structure of the pad.

Preferably, the sanitary napkin of the present invention is a single-use, disposable pad.

Preferably, the sanitary napkin of the present invention is presented in the form of a kit, and comprises a top sheet comprising or consisting of an antimicrobial metal.

Preferably, the sanitary napkin of the present invention is in the form of a kit, and the kit comprises a plurality of sanitary napkins, wherein a portion of the sanitary napkins is provided with an antimicrobial metal-based fiber and/or a top sheet having the same effect, while another portion of the sanitary napkins is provided with a material that has a beneficial, regenerating effect to the skin, e.g. a probiotic material and/or a top sheet having the same effect and optionally comprises a top sheet comprising or consisting of an antimicrobial metal.

In another preferred embodiment, the invention further relates to a diaper comprising
an absorbent diaper pad body having
an inner side in contact with the user's skin surface (surface in contact with an intimate body surface), and
an outer side, and
in the diaper pad body, a flexible absorbent fibrous unit is disposed between its inner and outer sides,
   characterized in that the diaper pad body comprises an antimicrobial metal-based fiber, wherein at least a part of the inner surface in contact with the user's skin surface is provided with an antimicrobial metal-based fiber, and/or

the antimicrobial metal-based fiber is arranged near the surface in contact with the skin surface, at a distance of not more than 30%, 25% or 20%, preferably not more than 15%, 10% or 5% of the total thickness of the product, preferably in case of an ellipsoid cross-section of its diameter, and/or

preferably, the antimicrobial metal-based fiber is arranged near, substantially parallel to, the surface in contact with the intimate body surface.

In another preferred embodiment, the invention further relates to a diaper comprising
an absorbent diaper pad body having
an inner side in contact with the user's skin surface, and
an outer side, and
a flexible absorbent fibrous unit disposed between its inner and outer sides,
   characterized in that at least a part of the inner surface of the diaper pad body that is in contact with the user's skin surface is provided with an antimicrobial metal-based fiber or metal particles,
   preferably, the antimicrobial metal-based fiber or metal particle has a biocidal, virucidal, bactericidal and/or fungicidal effect or a combination thereof.

Preferably, the antimicrobial metal-based fiber is arranged near, substantially parallel to, the surface in contact with the intimate body surface.

In the diaper according to the invention, the antibacterial metal-based fiber is in the form of a spiral, wavy or zigzag shape.

Preferably, the invention relates to a diaper comprising an absorbent pad body having
an inner side in contact with the user's skin surface, and
an outer side, and
a flexible absorbent fibrous unit disposed between the inner and outer sides of the absorbent pad body, wherein one side and a part of the fibrous unit form a first part, and
   the other side and the other part of the fibrous unit form a second part,
   characterized in that the first part is provided at least partly with an antimicrobial metal-based fiber, and the second part is made of a fabric material which is permeable to metal particles, e.g. for metal ions, metal atoms or nanoparticles derived from the fibers arranged in its structure.
   The diaper according to the invention, wherein the first part is arranged on the inner side and they are formed as a unitary part, and the second part is arranged on the outer side and they are formed as a unitary part.

Preferably, in the diaper of the present invention, the antibacterial metal-based fiber is in the form of a spiral, wavy or zigzag shape.

Preferably, in the diaper of the present invention, the antimicrobial material is inserted into the absorbent fiber structure of the diaper.

Preferably, the diaper of the present invention is a single-use, disposable diaper.

Preferably, the diaper of the present invention is in the form of a kit, and comprises a top sheet comprising or consisting of an antimicrobial metal.

Preferably, the diaper of the present invention is the diaper shown in FIG. 13 (P1) or 14 (P2).

Preferably, the diaper of the present invention is in the form of a kit, and the kit comprises a plurality of diapers, wherein a portion of the diapers is provided with an antimicrobial metal-based fiber and/or a top sheet having the same effect, while another portion of the diapers is provided with a material that has a beneficial, regenerating effect to the skin, e.g. a probiotic material and/or a top sheet having the same effect and optionally comprises a top sheet comprising or consisting of an antimicrobial metal.

In a preferred embodiment, the antimicrobial metal-based fiber in the pad of the invention is a metal fiber.

In another preferred embodiment, at least the antimicrobial fiber in the diaper is a fiber containing metal particles that comprise a non-metallic carrier and metal particles in or on the surface thereof, wherein the metal particles are preferably metal ions, metal atoms or nanoparticles, i.e. the metal is present in colloidal form. Preferably, the non-metallic carrier fiber is impregnated with a solution or suspension of the metal particles. The non-metallic carrier is preferably a fiber made of a biologically tolerable, absorbent material compatible with the use of the diaper, preferably of plant origin, e.g. bombast, cotton, or synthetic fiber, e.g. polyester, or fiber of animal origin, e.g. wool. Preferably, the metal is selected from the following materials: silver, copper, zinc, gold, palladium, platinum, aluminum, nickel, cobalt, silicon, calcium, titanium, chromium, molybdenum, vanadium, tungsten, gallium, selenium, tin, tellurium and/or a combination thereof.

Definitions

As used herein, the term “antimicrobial” effect, refers to an antibacterial (against bacteria), preferably bactericidal (bacteria-killing), anti-fungal (against fungi), preferably fungicidal (fungi-killing), and antiviral (against viruses), preferably virucidal (virus-killing, more specifically virus-inactivating) effect. The “antimicrobial” effect is preferably at least an antibacterial and/or antifungal, preferably bactericidal and/or fungicidal effect.

The term “metal-based” includes metal or any physical or chemical form of metals from which metal particles are released so that they can have an antimicrobial effect in a fluid in contact with a living body, preferably a body fluid.

As used herein, the term “metal particle” refers to any chemical entity containing the nucleus of a metal element in the periodic table, preferably an atom, ion or nanoparticles comprising thereof of the element, which is capable of displacing in solution by diffusion, thus metal particles can dissolve from the antimicrobial fiber according to the invention and have an antimicrobial effect at a desired location.

As used herein, the term “fiber” refers to a flexible (bendable and pliable) structure having a length much (at least 1, preferably 2 orders of magnitude) greater than its diameter, which is preferably at most 2 mm, more preferably at most 1 mm.

As used herein, “sanitary napkin” or “sanitary pad” includes any absorbent pad in contact with a body surface, such as a sanitary towel and pantyliner and an incontinence pad.

The term “toxic” means that the certain substance inhibits the growth of a microorganism of interest, preferably rendering the microorganism of interest incapable of reproducing, preferably harmful microorganisms. “Microorganisms” include viruses in a broad sense, thus include e.g. viruses, fungi, bacteria. Preferably, toxic to a microorganism means antimicrobial. Also preferably it means e.g. a biocidal, virucidal, fungicidal, bactericidal effect. In a highly preferred embodiment, the toxic substance according to the invention is toxic only to a microorganism harmful to humans or mammals.

### BRIEF DESCRIPTION OF THE FIGURES

FIG. 1 is a perspective view of a vaginal tampon according to an embodiment, wherein the tampon is provided with a groove parallel to its longitudinal axis along its entire surface.

FIG. 2 is a perspective view of a vaginal tampon according to an embodiment, wherein the tampon is provided with a continuously circumferential oblique groove along its entire surface.

FIG. 3 is a perspective view of a vaginal tampon according to an embodiment, wherein the tampon is provided with a continuously circumferential oblique groove along its entire surface, wherein said groove is more densely formed near the insertion end.

FIG. 4 is a schematic lateral cross sectional view of a vaginal tampon according to an embodiment, wherein an additional antimicrobial fiber is disposed within the absorbent fibrous unit of the tampon.

FIG. 5 is a schematic lateral cross sectional view of a vaginal tampon according to an embodiment having an absorbent fibrous unit comprising a plurality of antimicrobial fibers and a plurality of antimicrobial materials.

FIG. 6 is a perspective view of a vaginal tampon according to an embodiment, which comprises an antimicrobial top sheet and/or a probiotic top sheet.

The rib of the tampon shown in FIG. 7a (T1a) is a plurality of longitudinal grooves formed along the entire circumference of the tampon body which is configured to receive a thin fiber having an antimicrobial effect. The fiber is arranged on the outer surface of the tampon on at least a part of the shaped ribs.

On the surface of the tampon shown in FIG. 7b (T1b) or in its material, the fibers extending near the inner side of the surface extend in a helical shape.

On the surface of the tampon shown in FIG. 7c (T1c) or in its material, the density of fibers extending helically near the inner side of the surface is non-uniform.

In the embodiment shown in FIG. 8a (T2a), the tampon comprises an inner part on which a metal-based antimicrobial fiber is arranged so that the fiber extends at least a part of the tampon body.

FIG. 8b (T2b) illustrates that the fibers inside the tampon may be in a spiral, wavy, or zigzag shape.

In the embodiment shown in FIG. 9 (T3), the tampon body is provided with an outer top sheet.

In the embodiment shown in FIG. 10 (T4), the fibers are helically arranged below the surface, extending densely side by side.

FIGS. 11 (B1) show an embodiment in which the inner surface of the pad in contact with the skin surface of the user is provided with an antimicrobial metal-based fiber. FIG. 11a (B1a) shows a sanitary napkin with wings, FIG. 11b (B1b) shows a wingless embodiment.

FIGS. 12 (B2) show sanitary napkins that comprise an inner part, which is provided at least partly with an antimicrobial metal-based fiber, and an outer part, which is made of a material, which is permeable to metal particles, e.g. for metal ions, metal atoms or nanoparticles derived from the fibers arranged in its structure. FIG. 12a (B2a) shows a sanitary napkin with wings, FIG. 12b (B2b) shows a wingless embodiment.

The diaper shown in FIG. 13 (P1) does not include a separate insert, the antimicrobial fiber is integrated into the surface of the diaper.

The diaper shown in FIG. 14 (P2) comprises an inner part, which is provided at least partly with an antimicrobial metal-based fiber, and an outer part, which is made of a material, which is configured to be permeable to metal particles, e.g. for metal ions, metal atoms or nanoparticles derived from the fibers arranged in its structure.

### DETAILED DESCRIPTION OF THE INVENTION

After application of the absorbent intimate hygiene product of the present invention, its absorbent fibers collect the liquid as the pad body expands.

A metal-based material with antimicrobial, biocidal, bactericidal, virucidal and/or fungicidal activity is used in the product according to the invention. Metals with antimicrobial properties can be toxic to microorganisms (microbes) of interest, such as for bacteria and/or fungi. Different metals can act on different microorganisms by different mechanisms.

The use of a particular type of metal fiber is justified by the fact that there are a number of metal particles (e.g. metal ions, metal atoms, nanoparticles) that are toxic to microorganisms (microbes), e.g. for bacteria and/or fungi. Different metals can act by different mechanisms. After insertion, a tampon provided with said antimicrobial metal-based substance(s) is capable of preventing possible infections in the vagina, such as toxic shock syndrome (TSS), bacterial vaginosis (BV), vaginal yeast infections, and for preventing cervical lesions, wounds, uterin wounds, epithelial lesions due to the infections. Optionally, the tampon used may be suitable for forming an anti-biofilm on the epithelial wound to isolate and protect the wound from external irritants, thereby promoting the process of scar formation and wound healing. Furthermore, the antimicrobial materials may be suitable for both the treatment of pre-existing infections, primarily in combination therapy, and for post-infection treatment.

The material used can be any material with antimicrobial activity, especially a metal-based material. These materials include, but are not limited to, silver, copper, zinc, gold, palladium, platinum, aluminum, nickel, cobalt, silicon, calcium, titanium, chromium, molybdenum, vanadium, tungsten, gallium, selenium, tin, tellurium and any combination thereof.

The materials used may also be any chemically bonded antimicrobial form of these metals and antimicrobial combinations thereof.

These include, in particular, as a preferred solution, metal fibers.

Another preferred solution is a fiber impregnated with metal particles, the material of which may be, without limitation, any absorbent fiber compatible with the fabric of the tampon, such as a fiber of plant origin, e.g. fiber formed of bombast, cotton, polysaccharides, or fiber of animal origin, e.g. wool, or synthetic fibers, e.g. polyester, or a mixture thereof, impregnated with a solution containing the metal particle, e.g. with a solution of a metal salt, a solution of a metal ion or a metal atom in a chemical complex, or nanoparticles, e.g. a suspension of colloidal metal.

In a particularly preferred embodiment, the fiber comprises silver colloid.

The preparation of metal particles is known to a person skilled in the art. Such solutions are described e.g. in the following patent documents:

US20030180511 discloses a process for preparing a fine particle dispersion from precious metals, as a result of which the primary particles first form agglomerates in the form of chains and then form spherical particles stabilized by dispersion.

WO09/056401 discloses nanosized transition metal particles, preferably nanoplatelets. U.S. patent document US20080295646 discloses a heat treatment process for producing metal, preferably silver, nano-prisms which form a colloidal suspension and which have a predetermined thickness and a single-mode size distribution.

EP2411140B1 discloses the preparation of nanoplatelet-shaped transition metal particles in which a dispersing colloid is prepared with a reducing agent which is treated with peroxide. The patent also discloses that a number of metal ions, such as silver, copper, zinc, gold, nickel, iron, titanium, palladium or platinum, particularly silver, have antimicrobial activity. The material from which the metal ion is released over time may be an elemental metal or a metal compound.

We mention here that in the case of silver, relatively small amounts of Ag⁺ are released from the pure silver metal upon contact with moisture during the oxidation process so that the antimicrobial effect of the metallic silver is long-lasting. If a higher concentration is required in the short term, a fiber impregnated with silver salt or silver ion complex can be used.

However, the possible substances used are not limited to the above list, any substance having antimicrobial properties which can be formulated, alone or in combination with another substance, for the local prevention, treatment and/or post-treatment of infections can be used. The combination of several antimicrobial materials may be advantageous as it may be effective against a wider range of pathogens. The material used is preferably an alloy comprising at least one antimicrobial material. The use of alloys made of several antimicrobial materials is particularly advantageous. Such an alloy can be any feasible alloy of the metals listed above, e.g. metal alloys used in medical practice and surgery, e.g. variants of 316L alloy, and any combination or permutation of all of these.

These materials can be used on tampons, sanitary napkins and diapers to prevent, treat and/or post-treat an infection. For post-treatment, it is preferred to use a substance that recovers the flora of the intimate body surface, preferably a pre- or probiotic, highly preferably a probiotic substance.

It will be apparent to those skilled in the art that the examples of the invention are merely exemplary and that the idea of the invention may be embodied in a variety of tampons prepared in other ways.

### EMBODIMENTS

The tampon according to the invention can be prepared, for example, as follows.

By way of example, a conventional tampon preparation method known to those skilled in the art is used.

Preferably, the tampon is formed from a woven or nonwoven absorbent material by winding, during which metal fibers are disposed before or after the outer surface is formed, which fibers extend on the inner or outer area of the surface, substantially parallel to the area of the surface or a geometric surface fitted thereon. Optionally, the fibers are covered with an additional thin layer of absorbent material. Optionally, the fibers are woven or blended into the absorbent material.

Optionally, the final shape of the tampon is formed by compression. In this process step, grooves are preferably formed in the surface of the tampon and the antimicrobial fibers are contained therein.

In a highly preferred embodiment, the withdrawal fiber (conductive fiber) is an antimicrobial fiber. In a highly preferred embodiment, the withdrawal fiber is formed as a continuation of the other antimicrobial fibers. In this case, according to a preferred example, the withdrawal (conductive) fiber can be up to, preferably a maximum of, 30, 40 or 50 cm and is present in the tampon body for a considerable length or over the entire length of the tampon body (vagina), e.g. in a looped form.

In another highly preferred embodiment, the withdrawal fiber is arranged as a separate fiber.

Basic tampon preparation methods are described, for example, in U.S. Pat. No. 4,642,108A or 3,477,102A. patent document.

The sanitary napkin according to the invention can be produced, for example, as follows.

By way of example, a conventional sanitary napkin preparation method known to those skilled in the art is used.

Preferably, the sanitary napkin is formed from a woven or nonwoven, e.g. pressed absorbent material by layering and pressing, during which metal fibers are disposed before or after the formation of the surface in contact with the intimate body surface, which fibers extend on the inner or outer side of the surface, substantially parallel to the area of the surface or a geometric surface fitted thereon. Optionally, the fibers are covered with an additional thin layer of absorbent material. Optionally, the fibers are woven or blended into the absorbent material.

Optionally, the final shape of the pad is formed by compression. In a typical embodiment, the surface of the pad is covered with a cover layer that retains the absorbent layer. In another embodiment, grooves are formed in the surface of the pad and the antimicrobial fibers are placed therein.

Basic sanitary napkin preparation methods are described, for example, in WO1996000547A1 document.

The diaper according to the invention can be produced, for example, as follows. The production of the diaper according to the invention is geometrically similar to the production of the pad, in particular the incontinence pad, but the absorbent material is typically looser, less woven (in the case of a woven fabric), and/or a lower pressure is used during compression. Furthermore, the extension of the diaper is obviously larger.

Preferably, the diaper is formed from a woven or nonwoven, e.g. pressed absorbent material by layering and pressing, during which metal fibers are disposed before or after the formation of the surface in contact with the intimate body surface, which fibers extend on the inner or outer side of the surface, substantially parallel to the area of the surface (the inner side of the diaper) or a geometric surface fitted thereon. Optionally, the fibers are covered with an additional thin layer of absorbent material. Optionally, the fibers are woven or blended into the absorbent material.

Optionally, the final shape of the diaper is formed by compression. In a typical embodiment, the surface of the diaper is covered with a cover layer that retains the absorbent layer.

Basic diaper preparation methods are described, for example, in WO2009031359A1 or U.S. Pat. No. 4,801,345A patent document.

Tampons

After inserting the tampon in the vagina, its absorbent fibers collect the fluid, while the tampon body expands evenly to take the shape of the vagina. The tampons used today may be provided with ribs, preferably twisted ribs, which allow the liquid to be guided into the body of the tampon.

In another development of vaginal tampons, a metal-based material having antimicrobial, biocidal, bactericidal, virucidal and/or fungicidal activity may be used in the tampon. Possible embodiments of the vaginal tampon are described below.

1. In a preferred embodiment of the tampon, the tampon is ribbed. The ribs of the tampon are preferably a groove formed around the entire circumference of the tampon body, which is configured to receive a thin fiber having antimicrobial activity. In this case the fiber is arranged on the outer surface of the tampon, on at least a part of the shaped rib.

The fiber placed in the groove can be arranged according to different patterns on the outer surface of the tampon. The fiber may be guided along the entire outer circumference of the tampon body, for example in the form of a single continuous fiber or in the form of several fibers having a smaller length. The surface provided with the antimicrobial fiber may be more densely arranged in certain parts of the tampon body. For example, in the region of the tampon tip, which is located near the cervix when the tampon is inserted in the vagina, the surface more densely covered with the fiber may prevent certain pathogens that have multiplied in the vagina from entering the uterus through the cervix and spreading the infection. Optionally, the fiber may extend beyond the end opposite to the tampon tip and may be led out of the vagina together with the tampon withdrawal string. In this case, the antimicrobial effect of the metal-based material can be ensured along the entire length of the vagina.

In one embodiment, the fibers extend longitudinally on or near the surface of the tampon. [See FIG. 7a (T1a).] Preferably, the ribs of the tampon are formed by a plurality of longitudinal grooves formed along the entire circumference of the tampon body, in which or in part of which the fibers are arranged.

In a further embodiment, the fibers extending on the surface of the tampon or in its material, close to the inner side of the surface, extend helically, i.e. at an angle between 0° and 90°, preferably between 10° and 80°, to the longitudinal axis of the tampon. [See FIG. 7b (T1b)]. Preferably, the ribs of the tampon are formed by helical grooves formed along the entire circumference of the tampon body, i.e. the grooves are at an angle between 0° to 90°, preferably at an angle between 10° to 80°, to the longitudinal axis of the tampon, which carry the antimicrobial fiber. [See FIG. 7b (T1b).]

In one embodiment, the density of the helically extending fibers, which extend on the surface of the tampon or in its material near the inner side of the surface is non-uniform. [See FIG. 7c (T1c)].

The fiber used can be guided along a different pattern on the outer and/or inner surface of the tampon body, for example in a spiral, wavy, zigzag shape or any other shape or form [See FIGS. 7a (T1a), 7b (T1b) and 7c (T1c)]. The arrangement of the fiber may be determined on the one hand by the length and shape of the formed groove, however, the arrangement and length of the fiber does not necessarily correspond to the total length of the groove. The fiber can also be placed in a particular section of the groove, in which case its length is less than the length of the groove. If the dimensions of the groove allow, a single groove can be used to accommodate a thin, repeatedly wrapped around fiber, a larger thickness fiber, multiple thinner fibers, and any combination of the foregoing. In the case of several fibers, their material may be different, however, the fibers comprise at least one metal-based material with antimicrobial activity. If a single continuous fiber is arranged in the groove by leading it through the groove multiple times, the total length of the fiber is greater than the total length of the groove. However, if the fiber is placed in a groove, the total circumferential extent of the groove is in all cases equal to or greater than the circumferential extent of the fiber used, since the formation of the groove is a prerequisite for the arrangement of the fiber. [See FIG. 7c (T1c)].

In a preferred embodiment, the withdrawal fiber itself or the withdrawal fiber also has antimicrobial activity.

The fiber used can be fixed in the groove using various techniques, such as pressing, using adhesives, etc.

The tampon is preferably a single-use, disposable tampon.

2. In another preferred embodiment of the tampon, the tampon comprises an inner part on which a metal-based antimicrobial fiber is arranged such that the fiber extends at least a portion of the tampon body. [See FIG. 8a (T2a).] The fiber may be embedded in a groove formed in the inner part or may be fixed on the surface of the inner part. The arrangement and pattern of the fiber correspond to the constructions described in the first embodiment. The tampon further comprises an outer part that surrounds the inner part of the tampon body, thereby covering the groove provided with the fiber. The fibers inside the tampon can be of any arrangement (pattern), in particular straight, spiral, wavy or zigzag shaped. [See FIG. 8b (T2b)].

The outer part is essentially a top sheet, which may consist of a suitable material, such as a breathable textile material, a probiotic fabric, a fabric with an antimicrobial material, etc. However, the top sheet can be any fabric used in a hygiene product. The top sheet may be permeable in its entire surface or in a part of its surface, optionally in a specific pattern, to metal particles, e.g. for metal ions, metal atoms or nanoparticles releasing the inside of the tampon. [See FIG. 8b (T2b).]

The tampon is preferably a single-use, disposable tampon.

3. In a preferred embodiment of the tampon, the antimicrobial fiber forms the innermost part of the tampon body, the “inner core”. Preferably, the tampon body is provided with a bundle of fibers, wherein the material of the fibers may be the same or different, however, each of the fibers has an antimicrobial effect. The fiber is completely surrounded by a layer of absorbent material that is permeable to metal particles, e.g. for metal ions, metal atoms or nanoparticles derived from the fibers arranged in the tampon body. The tampon body may preferably be provided with ribs, which guide the liquid inside the tampon body, thereby facilitating the delivery of the metal particles, e.g. the dissolution of metal ions from the fiber.

The tampon body may also be provided with an outer top sheet, which may be any of the top sheets used in the previous embodiment. The top sheet is preferably a fabric with an antimicrobial material that is permeable to metal particles, e.g. for metal ions, metal atoms or nanoparticles. [See FIG. 9 (T3).]

The tampon is preferably a single-use, disposable tampon.

4. In a preferred embodiment of the tampon, the antimicrobial material is inserted into the absorbent fiber structure of the tampon. In this case, the material of the tampon comprises the antimicrobial material, from which the finished product is produced by pressing and/or by special molding (e.g. warping, folding, twisting). As a result of the liquid, the absorbent fibers swell and at the same time metal particles release from the inserted antimicrobial fibers, e.g. metal ions dissolve. The tampon body is designed to be permeable to metal particles, e.g. for metal ions, metal atoms or nanoparticles derived from the fibers arranged in its structure. In this case, the tampon body can preferably be provided with ribs, which guide the liquid inside the tampon body, thereby facilitating the delivery of the metal particles, e.g. dissolution of metal ions from fibers.

In an embodiment, the antimicrobial material is arranged on or near the inner surface of the top sheet or outer part of the tampon, and the tampon is preferably formed by twisting and/or pressing the absorbent material together with the antimicrobial material prior to or during the tampon forming operation.

In an embodiment, the antimicrobial material is disposed between the absorbent fibers, mixed therewith, to form a composite fabric, and the tampon is preferably formed so that the antimicrobial material, e.g. fibers are already provided in the base material, even before the twisting and/or pressing operation. Prior to twisting, the layers are arranged so that the antimicrobial fibers or a substantial portion thereof are brought close to the surface during twisting, preferably to extend under the surface. In a preferred embodiment, the fibers are arranged in a spiral shape below the surface, extending densely next to each other. In a highly preferred embodiment, the antimicrobial fiber is integrated or inserted in the fiber structure of the outer sheath. [See FIG. 10 (T4).]

The absorbent fabric applied with antimicrobial fibers can be any fabric used in a hygienic product, preferably a breathable textile fabric, a probiotic fabric, etc.

The tampon is preferably a single-use, disposable tampon.

5. The tampon can also be implemented in the form of a kit, which comprises, on the one hand, a vaginal tampon, which can be any commercially available or self-developed tampon, and, on the other hand, a top sheet comprising or consisting of an antimicrobial metal. The top sheet can be any commercially available or self-developed top sheet with antimicrobial activity. Both the tampon and the top sheet can be treated with a probiotic substance. The top sheet can be placed and fixed on the outer surface of the tampon body. The top sheet can be cut to a corresponding size and/or foldable. The kit may comprise tampons having different dimensions and top sheets having dimensions which correspond to dimensions of tampons. The assembled tampon is also suitable for the prevention, treatment and post-treatment of a vaginal infection. The tampon and top sheet are preferably single-use, disposable products.

6. Another embodiment is a kit that comprises vaginal tampons, which may be any commercially available or self-developed tampons, further comprises a top sheet comprising or consisting of an antimicrobial metal and a top sheet having a beneficial effect on the vaginal flora, e.g. probiotic effect. The top sheets can be any commercially available or self-developed top sheets with a target effect. The top sheets can be placed and fixed on the outer surface of the tampon body. Furthermore, the top sheets can be cut to size and/or foldable. The antimicrobial top sheet and the top sheet having the beneficial effect on the vaginal flora, e.g. the probiotic effect may have a different woven structure and/or packaging, making it easier for the user to select and apply the top sheet with the desired effect. Preferably, tampons with different top sheets can be cyclically changed by the user during a treatment period, thus ensuring the restoration and/or maintenance of the optimal vaginal flora. Optionally, top sheets with different effects can be applied to a tampon at the same time. The kit may comprise tampons having different dimensions and top sheets having dimensions which correspond to dimensions of tampons. The assembled tampon with the top sheet is suitable for the prevention, treatment and post-treatment of a vaginal infection. The tampons and top sheets are preferably single-use, disposable products.

7. Another embodiment is a kit comprising a plurality of vaginal tampons, wherein a portion of the tampons is provided with an antimicrobial metal-based fiber and/or a top sheet having a similar effect, while another portion of the tampons is provided with a material having a beneficial effect on the vaginal flora, e.g. probiotic effect and/or a probiotic top sheet. Tampons with different effects preferably have different packaging and/or external structures, resulting in a user-friendly kit assembly. Preferably, the tampons having different effects can be cyclically changed by the user during a treatment period, thus ensuring the restoration and/or maintenance of the optimal vaginal flora. The kit may comprise tampons of different sizes, which allows the selection of tampons of a size adapted to the user's menstrual cycle and/or to the user's anatomy. The tampon having a target effect is suitable for the prevention, treatment and post-treatment of a vaginal infection. The tampon is preferably a single-use, disposable tampon.

In one embodiment, the invention relates to the following types of tampons:

A preferred embodiment of the invention is a tampon, which comprises antimicrobial fibers in the grooves formed on the outer surface of the tampon body, thus being suitable for overcoming the problems described above.

The invention (optionally a design) is based on the recognition that if the antimicrobial metal fiber is directly contacted with the vaginal secretions, the dissolution of the metal particles and the restoring of the balance of the vaginal flora begins immediately after insertion.

The objects are achieved by providing a vaginal tampon for the prevention, treatment and/or post-treatment of an infection, wherein the tampon comprises:
a cylindrical tampon body having a circumference, length, longitudinal axis, insertion end and withdrawal end, wherein the tampon body comprises an absorbent, fibrous unit pressed into a self-supporting shape;
a withdrawal fiber connected to the tampon body near its withdrawal end;
   wherein the tampon body comprises at least one groove along the outer surface of the tampon body,
   wherein the at least one groove has a depth that is adapted to embed at least one antimicrobial fiber, wherein the at least one antimicrobial fiber is arranged in at least a portion of the at least one groove.

Preferably, the at least one antimicrobial fiber is a metal-based fiber comprising metal particles toxic to particular microorganisms, selected in particular from the following materials: silver, copper, zinc, gold, palladium, platinum, aluminum, nickel, cobalt, silicon, calcium, titanium, chromium, molybdenum, vanadium, tungsten, gallium, selenium, tin, tellurium and/or a combination thereof.

The metal particles preferably have biocidal, bactericidal, virucidal, fungicidal effect and/or any combination thereof.

Preferably, the diameter of the at least one antimicrobial fiber is about 0.05 mm to 4 mm, preferably between 0.1 mm and 4 mm, preferably between 0.5 mm and 3 mm, more preferably between 0.5 mm and 2 mm, most preferably between 0.5 mm and 1 mm or between 0.1 mm and 0.5 mm.

The at least one groove can be formed continuously, intermittently, in a spiral, zigzag or wavy shape (pattern).

The at least one groove preferably has a depth that is adapted to embed a plurality of antimicrobial fibers, wherein the material of the antimicrobial fibers varies.

The at least one groove preferably has a depth that is adapted to embed a plurality of antimicrobial fibers, wherein the material of the antimicrobial fibers is the same.

The at least one antimicrobial fiber preferably covers a larger portion of the outer surface of the tampon body near the insertion end than near the withdrawal end of the tampon body.

The at least one antimicrobial fiber, together with the withdrawal fiber, may be guided near the withdrawal end of the tampon body.

Preferably, the cylindrical tampon body has a length of about 3 cm to 15 cm, such as 4 cm to 15 cm, preferably about 3 cm to 11 cm, more preferably 4 cm to 8 cm or 3 cm to 6 cm, and a diameter of about 0.5 cm to 1.5 cm in the dry state, while its diameter in the wet state is between about 1 cm and 3 cm or between 1.5 cm and 4 cm, preferably about 2.5 cm.

The vaginal tampon is preferably provided with an applicator.

In a preferred embodiment, the outer surface of the tampon body may be provided with a top sheet which is permeable to the metal particles.

The top sheet is preferably a perforated fabric of breathable material, which is additionally provided with a probiotic and/or antimicrobial material.

The tampon body preferably comprises at least one antimicrobial fiber arranged on at least a portion of the length of the tampon body that is disposed inside the absorbent fibrous unit.

The tampon body may contain antimicrobial material(s) inside the absorbent fibrous unit.

In one embodiment, the object is further achieved by providing a tampon kit for the prevention, treatment and/or post-treatment of an infection, that comprises a vaginal tampon according to the invention and a top sheet comprising or consisting of an antimicrobial metal, wherein the top sheet covers at least a portion of the outer surface of a tampon body.

The top sheet is preferably a foldable and/or cuttable top sheet.

The tampon kit preferably comprises vaginal tampons having different dimensions and top sheets having different dimensions that cover at least a portion of the outer surface of the tampon of a size.

The objects are finally achieved by providing a tampon kit for the prevention, treatment and/or post-treatment of an infection, that comprises a plurality of vaginal tampons according to the invention, a top sheet comprising or consisting of an antimicrobial metal and a top sheet having a probiotic effect.

The top sheets are preferably foldable and/or cuttable top sheets.

The tampon kit may comprise tampons of different sizes.

The tampon kit preferably comprises vaginal tampons having different dimensions and top sheets having different dimensions that cover at least a portion of the outer surface of the tampon of a size.

The antimicrobial and probiotic top sheets preferably have a different pattern, structure, color and/or packaging.

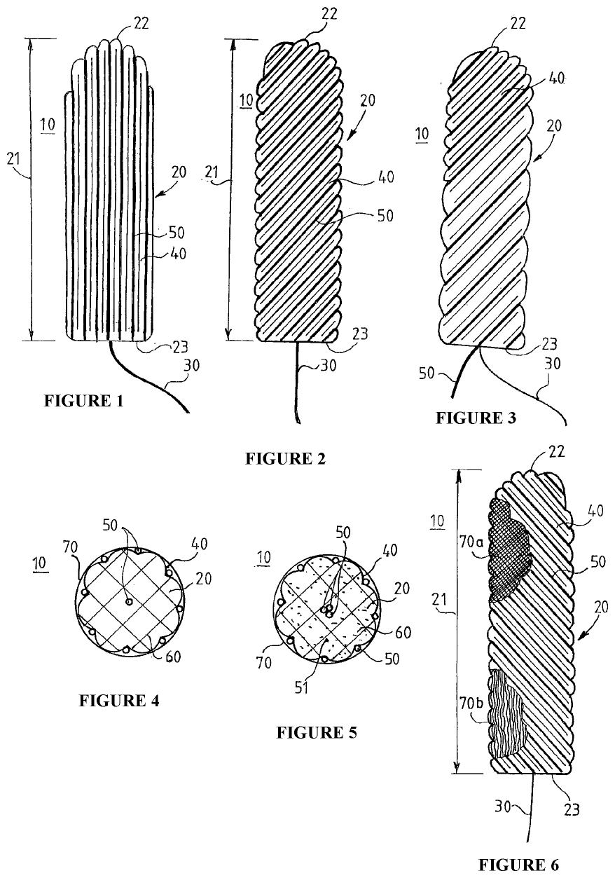

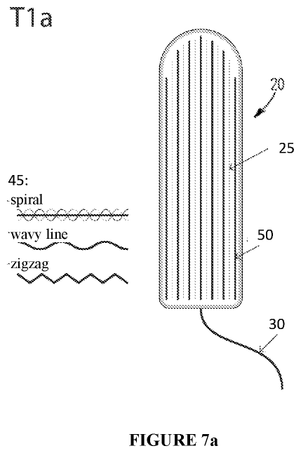

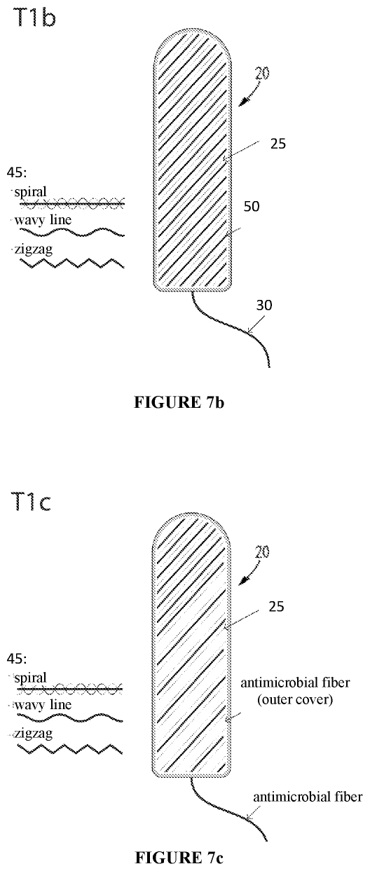

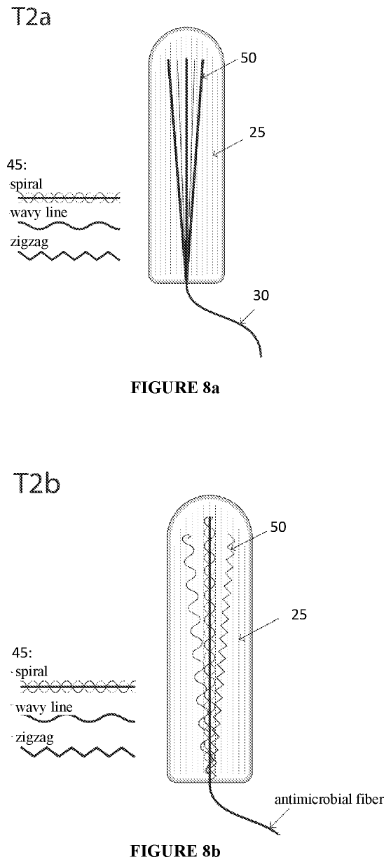

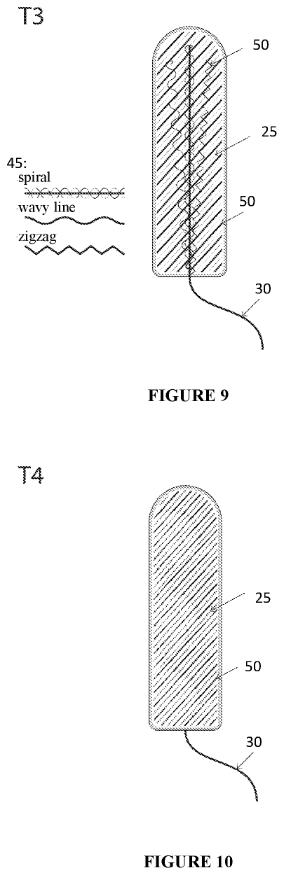

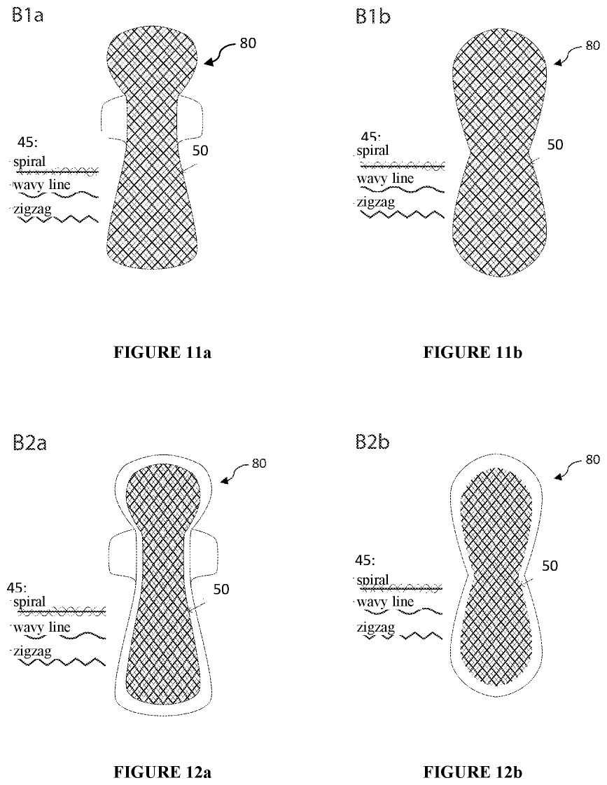

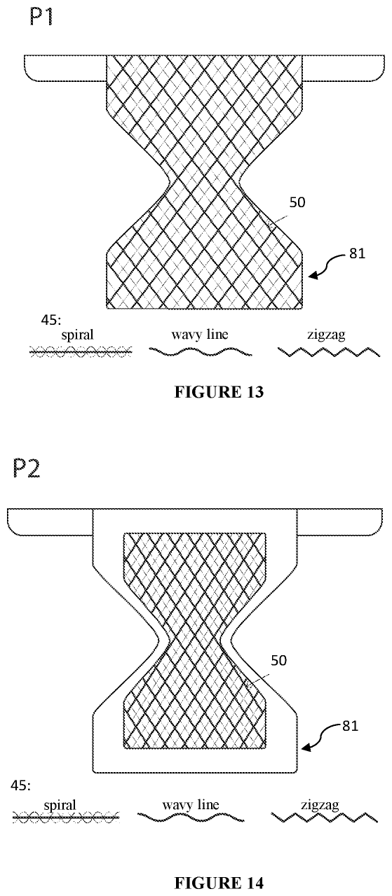

Furthermore, the solution is described in FIGS. 1 to 6 as an example. In the figures:
FIG. 1 is a perspective view of a possible embodiment of a vaginal tampon according to an embodiment, wherein the tampon is provided with a groove parallel to its longitudinal axis along its entire surface;
FIG. 2 is a perspective view of an embodiment of a vaginal tampon according to an embodiment, wherein the tampon is provided with a continuously circumferential oblique groove along its entire surface;
FIG. 3 is a perspective view of an embodiment of a vaginal tampon according to an embodiment, wherein the tampon is provided with a continuously circumferential oblique groove along its entire surface, which groove is more densely designed near the insertion end;
FIG. 4 is a schematic lateral cross sectional view of a vaginal tampon according to an embodiment, wherein an additional antimicrobial fiber is arranged inside the absorbent fibrous unit of the tampon;
FIG. 5 is a schematic lateral cross sectional view of a vaginal tampon according to an embodiment, wherein the absorbent fibrous unit comprises a plurality of antimicrobial fibers and a plurality of antimicrobial materials; and
FIG. 6 is a perspective view of a vaginal tampon according to an embodiment, which comprises an antimicrobial top sheet and/or a probiotic top sheet.

FIG. 1 shows a possible embodiment of a vaginal tampon 10 according to an embodiment. The tampon 10 comprises a cylindrical tampon body 20 having a circumference, length, and longitudinal axis 21. The tampon 10 has an insertion end 22 and a withdrawal end 23 opposite it. The tampon body 20 comprises an absorbent, fibrous unit 60 pressed into a self-supporting shape. The fibrous unit 60 is produced by a conventional compression process used in the art or an equivalent similar process, which is not described herein. The material of the fibrous unit 60 can be any fabric used in human hygiene products. The tampon 10 comprises a withdrawal fiber 30 connected to the tampon body 20 near its withdrawal end 23. The withdrawal fiber 30 allows the user to remove the tampon 10 applied during its use. The tampon body 20 of FIG. 1 comprises a plurality of grooves 40 formed along the entire outer surface of the tampon body 20 parallel to the longitudinal axis 21. The groove 40 has a depth suitable for embedding at least one antimicrobial fiber 50. Accordingly, the antimicrobial fiber 50 and the outer surface of the tampon body 20 are arranged in a plane. The tampon body 20 of FIG. 1 comprises an antimicrobial fiber 50 in each of the grooves 40 guided along its entire section, extending to the withdrawal end 23 and not extending beyond it. Optionally, the antimicrobial fiber 50 embedded in the groove 40 may be arranged in at least a portion of the groove 40, the representation of which is omitted herein. In case of the tampon 10 according to the embodiment, the extension of the at least one groove 40 is in each case greater than or equal to the extension of the at least one antimicrobial fiber 50. The antimicrobial fiber 50 can be fixed in the at least one groove 40 using various techniques, such as pressing, using adhesives, etc.

The material used as the antimicrobial fiber 50 can be any antimicrobial material, especially a metal-based material, which contains metal particles 51 which are toxic to certain microorganisms. These materials may include, but are not limited to, silver, copper, zinc, gold, palladium, platinum, aluminum, nickel, cobalt, silicon, calcium, titanium, chromium, molybdenum, vanadium, tungsten, gallium, selenium, tin, tellurium and any combination thereof. However, the possible materials used are not limited to the above list, any material having antimicrobial properties that can be formulated, alone or in combination with any other material, for the topical prevention, treatment and/or post-treatment of infections can be used.

The combination of several antimicrobial materials may be advantageous as it may be effective against a wider range of pathogens. The metal particles 51 have biocidal, bactericidal, virucidal, fungicidal effect and/or a combination thereof. The material used is preferably an alloy comprising at least one antimicrobial material. The use of alloys made of several antimicrobial materials may be particularly advantageous. Such an alloy may be any feasible alloy of the metals listed above, e.g., metal alloys used in medical practice and surgery, such as 316L alloy variants, and any combination or any permutation thereof.

Preferably, the metal used as the antimicrobial fiber 50 is non-irritating to the vaginal tissue, thus it can be used safely for medical applications.

The diameter of the antimicrobial fiber 50 is between about 0.5 mm and 4 mm, preferably about 2 mm. In the embodiment of the tampon 10 of FIG. 1, a single, thicker antimicrobial fiber 50 is disposed and fixed in the groove 40. Optionally, a plurality of thinner antimicrobial fibers 50 may be arranged and fixed in the at least one groove 40. In case of a plurality of thinner antimicrobial fibers 50, their materials may be different materials, however, at least one of the fibers 50 comprises at least one antimicrobial metal-based material. Tampons 10 with a plurality of antimicrobial fibers 50 are not shown in this document.

The antimicrobial fiber 50 used can be guided along the outer surface of the tampon body 20 in different patterns, such as continuously, intermittently, or in a spiral, wavy, zigzag shape, or any other shape (pattern) or form. The arrangement of the antimicrobial fiber 50 may be determined on the one hand by the length and shape of the groove formed, in which case the antimicrobial fiber 50 extends continuously in the at least one groove 40. However, the arrangement and length of the antimicrobial fiber 50 does not necessarily correspond to the total length of the at least one groove 40. The antimicrobial fiber 50 may also be arranged in a particular section or sections of the at least one groove 40, wherein the total length of the sections of the antimicrobial fiber 50 is less than the total length of the at least one groove 40.

If a single continuous antimicrobial fiber 50 is arranged in the at least one groove 40, it is arranged several times along it, the total length of the antimicrobial fiber 50 being greater than the total length of the at least one groove 40.

The vaginal tampon 10 is preferably a single-use, disposable tampon. The cylindrical tampon body 20 has a length of about 6 to 15 cm, preferably about 11 cm, and a diameter of about 0.5 cm to 1.5 cm in the dry state, and a diameter of about 1.5 cm to 4 cm, preferably about 2.5 cm in the wet state.

Furthermore, the tampon 10 of the embodiment may be provided with an applicator, wherein the tampon 10 is sterile encapsulated in the applicator prior to application. The applicator may be packaged with the received tampon 10. The tampon 10 provided with an applicator is not shown in this document.

FIG. 2 is a perspective view of another possible embodiment of a vaginal tampon 10 according to an embodiment.

The tampon 10 of FIG. 2 is provided with a continuous, circumferential groove 40 along its entire surface, which is inclined at an angle, in which groove 40 a continuous antimicrobial fiber 50 is arranged, as previously described.

The conformation of the groove 40 in the above figure also functions as a helical rib which facilitates the introduction of the liquid into the tampon body 20 and thereby the release of the metal particles 51 from the antimicrobial fiber 50.

The vaginal tampon 10 of FIG. 3 has an oblique groove 40 similar to the embodiment of FIG. 2, which is more densely formed near the insertion end 22 than near its withdrawal end 23. According to the design of the groove 40, the antimicrobial fiber 50 disposed therein covers a larger portion of the outer surface of the tampon body 20 near the insertion end 22 than near the withdrawal end 23 of the tampon body 20. In the case according to the figure, the antimicrobial fiber 50 extends beyond the withdrawal end 23 opposite the tampon tip and is led out from the vagina together with the tampon withdrawal fiber 30, which is for removing the tampon 10. In this case, the antimicrobial effect of the metal-based material is ensured along the entire length of the vagina.

In the inserted state of the tampon 10 of the above preferred embodiment, the end of the tampon body 20 more densely covered with antimicrobial fiber 50 is located near the cervix, thereby preventing certain pathogens that have multiplied in the vagina from entering the uterus through the cervix and spreading the infection.

FIG. 4 schematically illustrates a lateral cross-section of a vaginal tampon 10 according to an embodiment. In another preferred embodiment of the tampon 10 of the invention, the tampon 10 comprises, in addition to the antimicrobial fiber 50 disposed in the groove 40 on its outer surface, an antimicrobial fiber 50 disposed at least a portion of the length of the tampon body 20 arranged within the absorbent fibrous unit 60. The inner antimicrobial fiber 50 may be embedded in the groove formed in the inner part or attached to the surface of the inner part. In a preferred embodiment of the tampon 10, the outer surface of the tampon body 20 may be provided with a top sheet 70 that is permeable to the metal particles 51.

The top sheet 70 essentially serves as a top layer, which may be comprised of a particular material, such as a breathable textile fabric, a probiotic fabric, a fabric with an antimicrobial effect, etc. However, the top sheet 70 can be any fabric used in a hygiene product. The top sheet 70 is permeable in its entire surface or in a part of its surface, optionally in a specific pattern, to metal particles, e.g. for metal ions, metal atoms or nanoparticles releasing the inside of the tampon 10. The top sheet 70 may be a perforated fabric of breathable material that is additionally provided with a probiotic and/or antimicrobial material.

FIG. 5 schematically illustrates a lateral cross-section of a vaginal tampon 10 according to an embodiment. In this embodiment of the tampon 10, the absorbent fibrous unit 60 comprises a plurality of antimicrobial fibers 50 and a plurality of antimicrobial materials. In this case, the antimicrobial fibers 50 form the innermost part of the tampon body 20, the “inner core”. Preferably, a bundle of antimicrobial fibers 50 is disposed in the tampon body 20, wherein the material of the fibers may be the same or different, however, at least one of the fibers has an antimicrobial effect. The bundle of antimicrobial fibers 50 is completely surrounded by a unit 60 of absorbent material that is permeable to metal particles 51, e.g. for metal ions, metal atoms or nanoparticles. The bundle of antimicrobial fibers 50 can be fixed in the absorbent unit 60 as described in the embodiment of FIG. 4. The tampon 10 of FIG. 5 further comprises antimicrobial material(s) 51 in the absorbent fibrous unit 60, preferably metal particles, applied to or between the fibers of the fibrous unit 60. The base material of the absorbent fibrous unit 60 is formed by twisting and/or pressing together with the antimicrobial material(s) prior to or during the forming operation of the tampon 10.

Preferably, the at least one groove 40 in the tampon body 20 may also serve as a rib, which may guide the liquid into the interior part of the tampon body 20, thereby also facilitating in the delivery of the metal particles 51, e.g. dissolution of the metal ions from the antimicrobial fiber 50 or from the antimicrobial material(s) in the fibrous unit 60.

The tampon body 20 of FIG. 5 may further be provided with a top sheet 70, which may be any of the top sheets 70 used in the previous embodiment. The top sheet 70 is preferably a fabric provided with an antimicrobial material that is permeable to metal particles, e.g. for metal ions, metal atoms or nanoparticles. The top sheet 70 may additionally be provided with additional probiotic and/or antimicrobial materials.

FIG. 6 is a perspective view of a vaginal tampon 10 according to an embodiment, which comprises an antimicrobial top sheet 70a and/or a probiotic top sheet 70b connected to the outer surface of the tampon body 20.

The tampon 10 of FIG. 6 is a ready-to-use tampon 10 assembled from a tampon kit according to the invention, wherein the tampon kit comprises a vaginal tampon 10 provided with an antimicrobial fiber 50, an antimicrobial top sheet 70a and a probiotic top sheet 70b. The antimicrobial top sheet 70a is toxic to certain microorganisms, thus enhancing the antimicrobial effect of the assembled tampon 10 including the top sheet 70a and the tampon 10. The probiotic top sheet 70b is used to create a balance condition in the vaginal area that has a beneficial effect on the vaginal flora.

One of the simplest embodiments of the tampon kit of the present invention comprises a vaginal tampon 10 according to the invention and a top sheet 70a comprising or consisting of an antimicrobial metal, wherein the top sheet 70a covers at least a portion of the outer surface of at least one tampon body 20.

The top sheet 70a, 70b in the tampon kit of FIG. 6 can be any commercially available or self-developed top sheet. The top sheet 70a, 70b is a foldable and/or cut-to-size top sheet that can be placed and attached to at least a portion of the outer surface, preferably to the entire outer surface of the tampon body 20. The size of the top sheet 70a, 70b preferably corresponds to the dimensions of the entire outer surface of the tampon body 20.

The tampon kit of the present invention may comprise tampons 10 having different dimensions and top sheets 70a, 70b having different dimensions so that cover at least a portion of the outer surface of the tampon 10 having desired size. Preferably, the dimensions of the top sheet 70a, 70b correspond to the dimensions of the tampon body 20 of the tampon 10 having desired size so that it is not necessary to cut the top sheet 70a, 70b to desired size.

The assembled 10 tampon is suitable for the prevention, treatment and post-treatment of a vaginal infection. The tampon 10 and top sheets 70a, 70b are preferably single-use, disposable products.

The tampon 10 according to FIG. 6, the antimicrobial top sheet 70a and the probiotic top sheet 70b having a beneficial effect on the vaginal flora may have different patterns, structures, colors, and/or packaging, making it easier for the user to select and apply the top sheet 70a, 70b with the desired effect. Preferably, the tampons 10 provided with the different effect top sheets 70a, 70b can be cyclically changed by the user during a treatment period, thus ensuring the restoration and/or maintenance of the optimal vaginal flora. Optionally, the top sheets 70a, 70b having different effects may be applied to a single tampon 10 at the same time. The tampon kit may comprise tampons 10 having different dimensions and top sheets 70a, 70b having dimensions corresponding to the dimensions of the tampon 10 which is selected for application. The assembled tampons 10 provided with the top sheet(s) 70a, 70b are suitable for the prevention, treatment and post-treatment of a vaginal infection. The tampon 10 and top sheets 70a, 70b are preferably single-use, disposable product elements.

### Sanitary Napkins

The aforementioned antimicrobial metal-based fiber can also be used in sanitary towels, pantyliners and incontinence pads, wherein its role is to prevent, treat and post-treat an infection in the genital areas, optionally the prevention, treatment and post-treatment of an infection in the vagina and surrounding areas, similar to the aforementioned vaginal tampon.

Possible embodiments of the sanitary napkin are described below.

1. In a preferred embodiment of the sanitary napkin, at least a portion of the inner surface of the pad body 80 in contact with the user's skin surface is provided with an antimicrobial metal-based fiber 50 (antimicrobial fiber). The arrangement and pattern of the fiber can be realized in a particular way. The fiber, which is used, can be attached to the surface of the pad using various techniques, for example by pressing and gluing. Preferably, the fiber may be positioned on the surface of the pad such that the fiber-coated and fiberless surfaces of the pad are in a plane. Furthermore, the fiber can be positioned so that the fiber protrudes from the inner surface of the pad in a dimension equal to its thickness, whereby it forms the innermost layer of the pad. According to an alternative embodiment which is advantageous according to a convenience and hygiene aspect, the pad is configured in a disposable form. In another embodiment, the pad is a reusable product that can be washed at high temperatures and can be tumble-dried. The latter variant can be environmentally beneficial. [See FIGS. 11a (B1a) and 11b (B1b).]

2. In another embodiment of the sanitary napkin, the pad body 80 comprises an inner part that is at least partially provided with an antimicrobial metal-based fiber 50 (antimicrobial fiber), and an outer part that is such a fabric material that is permeable to metal particles, e.g. for metal ions, metal atoms or nanoparticles derived from the fibers arranged in the structure of the sanitary napkin. When the pad is used, the inner part is directly contacted with the user's skin surface. The outer part is indirectly contacted with the user's skin surface can be of any fabric used in a hygienic product, such as a breathable textile fabric, a probiotic fabric, a fabric with an antimicrobial material, etc. A preferred construction of the pad is a single-use, disposable pad. Another embodiment of the sanitary napkin is a reusable product that can be washed at high temperatures and can be tumble-dried. [See FIGS. 12a (B2a) and 12b (B2b).]

3. In another preferred embodiment of the sanitary napkin, the antimicrobial material is inserted into the absorbent fiber structure of the pad body. As a result of the liquid, the absorbent fibers swell and at the same time metal particles release from the applied antimicrobial fibers 50, e.g. metal ions dissolve. The fiber structure is designed so that it is permeable to metal particles, e.g. for metal ions, metal atoms or nanoparticles. The material of the absorbent fiber structure may be any fabric used in a hygienic product, which may optionally be provided with a probiotic material.

In a variant of the pad, the antimicrobial material is arranged on or near the inner surface of the top sheet or outer part of the sanitary napkin, and the pad is preferably formed by twisting and/or pressing the absorbent base material together with the antimicrobial material prior to or during the pad body 80 forming operation.

In another variant, the antimicrobial material is disposed between the absorbent fibers, mixed therewith, to form a composite fabric, and the pad is preferably formed so that the antimicrobial material, e.g. fibers are already provided in the base material, even before the twisting and/or pressing operation.

A preferred variant of the pad is a single-use, disposable pad. Another variant of the sanitary napkin is preferably a reusable product which can be washed at high temperature and can be tumble-dried.

4. The sanitary napkin may also be in the form of a kit, that comprises, on the one hand, a sanitary napkin, which may be any commercially available or self-developed sanitary napkin, and, on the other hand, a top sheet comprising or consisting of an antimicrobial metal. The top sheet can also be any commercially available or self-developed antimicrobial top sheet. Optionally, the pad or the top sheet may be provided with a material having a beneficial, regenerating effect on the skin surface, e.g. with a probiotic material. The fiber structure of the pad is preferably breathable, and may be perforated on at least a portion of its surface. The top sheet can be placed and attached on the inner surface of the pad. The top sheet can be cut to size and/or foldable. The kit may comprise pads having different dimensions and top sheets having dimensions corresponding to the dimensions of the pad which is selected for application. A preferred variant of the pad is a single-use, disposable pad. Another variant of the pad is a reusable product which can be washed at high temperatures and can be tumble-dried. The assembled sanitary napkin is also suitable for the prevention, treatment and post-treatment of an infection.

5. Another embodiment is a kit comprising a sanitary napkin, which may be any commercially available or self-developed sanitary napkin, further comprising a top sheet comprising or consisting of an antimicrobial metal and a top sheet having a beneficial, regenerating effect on the skin surface, e.g. a probiotic top sheet. The specific top sheet can be any commercially available or self-developed top sheet with the defined effect. The top sheets can be placed and attached on the inner surface of the pad. Furthermore, the top sheets can be cut to size and/or foldable. The top sheet with the antimicrobial effect and the top sheet having the beneficial, regenerating effect on the skin surface, e.g. the probiotic top sheet may preferably be of a different weave and/or packaging, which facilitates the user to select and apply the top sheet with the desired effect. Preferably, the pads with the different effect top sheet can be cyclically changed by the user during a treatment period. Optionally, the top sheets with different effects can be applied to a sanitary napkin simultaneously. The kit may comprise pads having different dimensions and top sheets having dimensions corresponding to the dimensions of the pad which is selected for application. A preferred variant of the pad is a single-use, disposable pad. Another variant of the pad is a reusable product that can be washed at high temperatures and can be tumble-dried. The assembled pad provided with the specific top sheet is suitable for the prevention, treatment and post-treatment of an infection.

6. Another embodiment is a kit comprising a plurality of sanitary napkins, wherein a portion of the sanitary napkins is provided with an antimicrobial metal-based fiber and/or a top sheet having a similar effect, while another portion of the sanitary napkins is provided with a material having a beneficial, regenerating effect on the skin surface, e.g. with a probiotic material and/or with a top sheet having a same effect. The sanitary napkins preferably have different packaging and/or external structure, resulting in a user-friendly kit assembly. Preferably, the pads with different effects can be cyclically changed by the user during a treatment period. The kit may comprise pads of different sizes. A preferred variant of the pad is a single-use, disposable pad. Another variant of the pad is a reusable product that can be washed at high temperatures and can be tumble-dried. The pad with the specific effect is suitable for the prevention, treatment and post-treatment of infections.

### Diaper

Possible embodiments of the children's diaper and the adult diaper are described below.

1. In a preferred embodiment of a children's diaper or an adult diaper, at least a portion of the inner surface of the diaper pad body 81 in contact with the user's skin surface is provided with an antimicrobial metal-based fiber 50 (antimicrobial fiber). In this case, the diaper does not contain a separate insert, the antimicrobial fiber is integrated into the surface of the diaper. The arrangement and pattern of the fiber can be implemented in any way. The fiber, which is used, can be attached to the surface of the diaper using various techniques, for example by pressing and gluing. Preferably, the fiber can be arranged on the surface of the diaper such that the fiber-coated and fiberless surfaces of the diaper are in a plane. Furthermore, the fiber can be positioned so that the fiber protrudes from the inner surface of the diaper in a dimension equal to its thickness, in which case it forms the innermost layer of the diaper which is directly contacted with the skin surface. The diaper may be a slip-on diaper or a diaper that is detachable on at least one side, such as a diaper that can be releasably attached by snaps or other means. The diaper is preferably configured as a liquid-sealed diaper. The diaper can be of different sizes. A preferred variant of the diaper is a single-use, disposable diaper. Another variant of the diaper is a reusable product that can be washed at high temperatures and can be tumble-dried. The diaper has dimensions such that the user's movement is not or only slightly obstructed, and is preferably completely unnoticeable under the garment during use. [See FIG. 13 (P1).]

2. In another embodiment of a children's diaper or an adult diaper, in the diaper, the diaper pad body comprises an inner part that is at least partially provided with an antimicrobial metal-based fiber, and an outer part that is a fabric material that is configured to be permeable to metal particles, e.g. for metal ions, metal atoms or nanoparticles derived from the fibers arranged in its structure. When the diaper is used, the inner part is directly contacted with the user's skin surface. The outer part is indirectly contacted with the skin surface, can be of any fabric used in a hygienic product, for example a breathable textile material, a fabric having a beneficial, regenerating effect on the skin surface, e.g. a probiotic fabric, a fabric provided with an antimicrobial material, etc. Both the inner part and the outer part are formed in one piece with the diaper. The diaper may be a slip-on diaper or a diaper that is detachable on at least one side, such as a diaper that can be releasably attached by snaps or other means. The diaper is preferably configured as a liquid-sealed diaper. The diaper can be of different sizes. A preferred variant of the diaper is a single-use, disposable diaper. Another variant of the diaper is a reusable product that can be washed at high temperatures and can be tumble-dried. The diaper has dimensions such that the user's movement is not or only slightly obstructed, and is preferably completely unnoticeable under the garment during use. [See FIG. 14 (P2).]

3. In a further preferred embodiment of a children's diaper or an adult diaper, the antimicrobial material is inserted to the absorbent fiber structure of the diaper pad body 81. As a result of the liquid, the absorbent fibers swell and at the same time metal particles release from the inserted antimicrobial fibers, e.g. metal ions dissolve. The fiber structure is designed to be permeable to dissolved metal particles, e.g. for metal ions, metal atoms or nanoparticles. The material of the absorbent fiber structure can be any fabric used in a hygienic product, which optionally has a beneficial, regenerating effect on the skin surface, e.g. may be provided with a probiotic material.

In a variant of the diaper, the antimicrobial material is arranged on the inner surface of the top sheet or the part of the diaper in contact with the skin surface, and the diaper is preferably formed by twisting and/or pressing the absorbent base material together with the antimicrobial material prior to or during the diaper forming operation.

In another variant, the antimicrobial material is disposed between the absorbent fibers, mixed therewith, to form a composite fabric, and the diaper is preferably formed so that the antimicrobial material, e.g. fibers are already provided in the base material, even before the twisting and/or pressing operation.

The diaper may be a slip-on diaper or a diaper that is detachable on at least one side, such as a diaper that can be releasably attached by snaps or other means. The diaper is preferably configured as a liquid-sealed diaper. The diaper can be of different sizes. A preferred variant of the diaper is a single-use, disposable diaper. Another variant of the diaper is a reusable product that can be washed at high temperatures and can be tumble-dried. The diaper has dimensions such that the user's movement is not or only slightly obstructed, and is preferably completely unnoticeable under the garment during use.

4. In a further preferred embodiment of a children's diaper or an adult diaper, the diaper comprises an insert (insert part) provided with an antimicrobial material, and a diaper (diaper part) suitable for receiving the insert and together forming the diaper pad body 81. The antimicrobial material may be integrated into the insert and/or applied to the surface of the insert. The material of the insert is permeable to metal particles, e.g. for metal ions, metal atoms or nanoparticles. The insert may optionally have a beneficial, regenerating effect on the skin surface, e.g. can also be treated with a probiotic substance. The insert can be fixed to the inside of the diaper, preferably with an adhesive applied to the surface opposite to the surface provided with the antimicrobial material. The diaper may be provided with a separate inner pocket to facilitate the fastening of the insert. The diaper is preferably configured as a liquid-sealed diaper, and may have at least partially absorbent fibers. The diaper may be a slip-on diaper or a diaper that is detachable on at least one side, such as a diaper that can be releasably attached by snaps or other means. The diaper and the insert, which is used for the diaper, have such dimensions that is suitable to assembly with each other, and can be produced in different sizes for users of different body shapes and/or body sizes. A preferred variant of the diaper and/or insert is designed to be disposable. Another variant of the diaper and/or insert is a reusable product that can be washed at high temperatures and can be tumble-dried. The diaper, as well as the diaper assembled with the insert, has dimensions such that the user's movement is not or only slightly obstructed and are preferably completely unnoticeable under the garment during use.

5. The diaper can also be implemented in the form of a kit, that comprises, on the one hand, a diaper for children or adults, which can be any commercially available or self-developed diaper, and, on the other hand, a top sheet comprising or consisting of an antimicrobial metal. The top sheet can also be any commercially available or self-developed antimicrobial top sheet. Optionally, the diaper may be provided with a material having a beneficial, regenerating effect on the skin surface, e.g. with a probiotic material. The fiber structure of the diaper is preferably breathable, and has perforations on at least a portion of its surface. The top sheet can be placed and attached to the outer surface of the diaper. The top sheet can be cut to size and/or foldable. The kit may comprise inserts having different dimensions and top sheets having dimensions corresponding to the dimensions of the insert which is selected for application, and may be produced in different sizes for users of different body shapes and sizes. The diaper may be a slip-on diaper or a diaper that is detachable on at least one side, such as a diaper that can be releasably attached by snaps or other means. The diaper is preferably configured as a liquid-sealed diaper. A preferred variant of the diaper or top sheet is disposable. Another variant of the diaper or top sheet is a reusable product that can be washed at high temperatures and can be tumble-dried. The diaper, as well as the diaper assembled with the top sheet, has dimensions such that the user's movement is not or only slightly obstructed, and are preferably completely unnoticeable under the garment during use. The assembled diaper is also suitable for the prevention, treatment and post-treatment of an infection.

6. Another embodiment is a kit comprising a diaper for children or adults, which may be any commercially available or self-developed sanitary diaper, further comprising a top sheet comprising or consisting of an antimicrobial metal and a top sheet having a beneficial, regenerating effect on the skin surface, e.g. a probiotic top sheet. The specific top sheet can be any commercially available or self-developed top sheet with a target effect. The top sheets can be placed and attached to the outer surface of the diaper. Furthermore, the top sheets can be cut to size and/or foldable. The top sheet with the antimicrobial effect and the top sheet having the beneficial, regenerating effect on the skin surface, e.g. the probiotic top sheet may preferably have a different woven structure and/or packaging, which facilitates the user in selecting and applying the top sheet with the desired effect. Preferably, diapers with different effect top sheets can be cyclically changed by the user during a treatment period. Optionally, top sheets with different effects can be applied to a diaper at the same time. The kit may comprise diapers having different dimensions and top sheets having dimensions corresponding to the dimensions of the diaper which is selected for application, and may be produced in different sizes for users of different body shapes and/or body sizes. The diaper may be a slip-on diaper or a diaper that is detachable on at least one side, such as a diaper that can be releasably attached by snaps or other means. The diaper is preferably configured as a liquid-sealed diaper. A preferred variant of the diaper or top sheet is disposable. Another variant of the diaper or top sheet is a reusable product that can be washed at high temperatures and can be tumble-dried. The diaper, as well as the diaper assembled with the top sheet, has dimensions such that the user's movement is not or only slightly obstructed, and is preferably completely unnoticeable under the garment during use. The assembled diaper is also suitable for the prevention, treatment and post-treatment of an infection.

7. Another embodiment is a kit comprising a plurality of diapers for children or adults, wherein a portion of the diapers is provided with an antimicrobial, metal-based fiber and/or a top sheet having the same effect, while another portion of the diapers is provided with a material having a beneficial, regenerative effect on the skin surface, e.g. with a probiotic material and/or provided with a probiotic top sheet. The material of the diaper is permeable to metal particles, e.g. for metal ions, metal atoms or nanoparticles. Preferably, the diapers have different packaging and/or external structures, resulting in a user-friendly kit assembly. Preferably, diapers with different effects can be cyclically changed by the user during a treatment period. The kit may comprise diapers having different sizes. The diaper may be a slip-on diaper or diaper that is detachable on at least one side, such as a diaper that can be releasably attached by snaps or other means. The diaper is preferably configured as a liquid-sealed diaper. A preferred variant of the diaper provided with a top sheet is disposable. Another variant of the diaper provided with a top sheet is a reusable product that can be washed at high temperatures and can be tumble-dried. The diaper has dimensions such that the user's movement is not or only slightly obstructed, and is preferably completely unnoticeable under the garment during use. The diaper having a target effect is suitable for the prevention, treatment and post-treatment of an infection.

### INDUSTRIAL APPLICABILITY

The invention can be preferably used in the field of absorbent intimate hygiene products because at least a part of the surface in contact with the intimate body surface is provided with an antimicrobial metal-based fiber, and thus, alone or in combination with other materials, is suitable for the local prevention, treatment and/or post-treatment of infections.

### LIST OF REFERENCE SYMBOLS

10 tampon
20 tampon body
22 insertion end
23 withdrawal end
25 outer part (outer cover)
30 withdrawal fiber
40 groove
45 pattern
50 antimicrobial fiber
51 metal particle
60 absorbent fibrous unit
70a, 70b top sheet
80 pad body
81 diaper pad body

1. An absorbent intimate hygiene product comprising an absorbent fibrous unit and an intimate body surface contacting side having a surface (intimate body surface contacting surface), wherein at least a part of the intimate body surface contacting surface is provided with at least one antimicrobial metal-based fiber having a diameter of about 0.05 mm to 4 mm, preferably a diameter of at most 2 mm, wherein metal particles are released from the metal-based fiber during use, which have antimicrobial, preferably biocidal, bactericidal, virucidal, fungicidal effect and/or a combination thereof
wherein, the antimicrobial metal-based fiber is arranged near to the intimate body surface-contacting surface, at a distance of not more than 30%, in proportion to the total thickness of the product, and is substantially parallel to the surface in contact with the intimate body surface
said product being a vaginal tampon.

2. The vaginal tampon according to according claim 1, wherein the fiber is
arranged near to the intimate body surface-contacting surface, at a distance of not more than 15%, preferably 10%, more preferably 5% in proportion to the total thickness of the product.

3. The vaginal tampon according to claim 1 wherein the fiber(s) is/are arranged on the outer side of the surface.

4. The vaginal tampon according to claim 1, wherein the fiber(s) is/are arranged on the inner side of the surface.

5. (canceled)

6. The vaginal tampon according to claim 1, wherein the fiber is a metal fiber, preferably a silver fiber and/or the fiber is an absorbent fiber compatible with the fabric of the tampon, said fiber being impregnated with metal particles, preferably silver particles.

7. (canceled)

8. The vaginal tampon according to claim 1, which is a vaginal tampon (10) comprising:
a cylindrical tampon body (20) having an insertion end (22) and a withdrawal end (23), wherein the tampon body (20) comprises an absorbent, fibrous unit (60) pressed into a self-supporting shape;
a withdrawal fiber (30) connected to the tampon body (20) near its withdrawal end (23);
wherein the tampon body (20) comprises at least one groove (40) along the outer surface of the tampon body (20),
characterized in that the at least one groove (40) has a depth that is adapted to embed the at least one antimicrobial fiber (50), wherein the at least one antimicrobial fiber (50) is arranged in at least a portion of the at least one groove (40), wherein preferably at least one groove (40) is formed in a continuous, intermittent, spiral, zigzag or wavy pattern.

9. The vaginal tampon (10) according to claim 1, characterized in that the at least one antimicrobial fiber (50) is a metal-based fiber comprising metal particles (51) toxic to microorganisms of interest
said antimicrobial fiber (50) comprising a non-metallic carrier and metal particles (51) in or on the surface thereof, wherein the metal particles (51) wherein the metal is present in colloidal form.

10. (canceled)

11. (canceled)

12. The vaginal tampon (10) according to claim 5, characterized in that the at least one groove (40) has a depth that is adapted to embed a plurality of antimicrobial fibers (50), wherein the material of the antimicrobial fibers (50) varies and/or is the same,
wherein the diameter of the at least one antimicrobial fiber (50) is between about 0.1 mm and 2 mm or between 0.5 mm and 4 mm.

13. (canceled)

14. (canceled)

15. The vaginal tampon (10) according to claim 8, characterized in that the at least one antimicrobial fiber (50) together with the withdrawal fiber (30) is guided near the withdrawal end (23) of the tampon body (20).

16. The vaginal tampon (10) according to claim 8, characterized in that the cylindrical tampon body (20) has a length of about 3 cm to 15 cm, preferably 3 cm to 11 cm, and a diameter of about 0.5 cm to 1.5 cm in the dry state, while its diameter in the wet state is between about 1.5 cm and 4 cm, preferably about 2.5 cm.

17. The vaginal tampon (10) according to claim 8, characterized in that it is provided with an applicator.

18. The vaginal tampon (10) according to claim 8, characterized in that the outer surface of the tampon body (20) is provided with a top sheet (70) which is permeable to the metal particles (51), wherein preferably the top sheet (70) is a perforated fabric of breathable material, which is, if desired, additionally provided with a probiotic and/or antimicrobial material.

19. (canceled)

20. The vaginal tampon (10) according to claim 8, characterized in that the tampon body (20) comprises antimicrobial material(s) inside the absorbent fibrous unit (60), preferably the tampon body (20) comprises at least one antimicrobial fiber (50) arranged on at least a part of the length of the tampon body (20), which is arranged inside the absorbent fibrous unit (60).

21. (canceled)

22. A kit for producing an absorbent intimate hygiene product according to claim 1, said product being a vaginal tampon, characterized in that it comprises a vaginal tampon (10) and an element comprising an antimicrobial metal-based fiber, preferably a top sheet (70a) comprising or consisting of a metal-based fiber, preferably a plurality of vaginal tampons (10), and a corresponding number of top sheets.

23. The tampon kit according to claim 22, characterized in that the absorbent intimate hygiene product is
an absorbent intimate hygiene product comprising an absorbent fibrous unit and an intimate body surface contacting side having a surface (intimate body surface contacting surface), wherein at least a part of the intimate body surface contacting surface is provided with at least one antimicrobial metal-based fiber having a diameter of about 0.05 mm to 4 mm, preferably a diameter of at most 2 mm, wherein metal particles are released from the metal-based fiber during use, which have antimicrobial, preferably biocidal, bactericidal, virucidal, fungicidal effect and/or a combination thereof
wherein, the antimicrobial metal-based fiber is arranged near to the intimate body surface-contacting surface, at a distance of not more than 30%, in proportion to the total thickness of the product, and is substantially parallel to the surface in contact with the intimate body surface
said product being a vaginal tampon;
wherein said vaginal tampon (10) comprises
a cylindrical tampon body (20) having an insertion end (22) and a withdrawal end (23), wherein the tampon body (20) comprises an absorbent, fibrous unit (60) pressed into a self-supporting shape;
a withdrawal fiber (30) connected to the tampon body (20) near its withdrawal end (23);
wherein the tampon body (20) comprises at least one groove (40) along the outer surface of the tampon body (20),
characterized in that the at least one groove (40) has a depth that is adapted to embed the at least one antimicrobial fiber (50), wherein the at least one antimicrobial fiber (50) is arranged in at least a portion of the at least one groove (40), wherein preferably at least one groove (40) is formed in a continuous, intermittent, spiral, zigzag or wavy pattern; wherein
the top sheet(s) (70a) are foldable and/or cuttable top sheets, and which is optionally free of antimicrobial fibers (50).

24. The tampon kit according to claim 22, characterized in that it comprises tampons (10) of different sizes, preferably it comprises vaginal tampons (10) having different dimensions and top sheet(s) (70a) having different dimensions, which cover at least a part of the outer surface of the tampon (10) of a size, and/or
the antimicrobial top sheet(s) (70a) have different patterns, structures, colors and/or packaging.

25-52. (canceled)

53. The tampon kit according claim 22, characterized in that it comprises probiotic top sheet and a vaginal tampon (10) and an element comprising an antimicrobial metal-based fiber, preferably a probiotic top sheet (70b), preferably a plurality of vaginal tampons (10), and a corresponding number of top sheets,
wherein the fiber is arranged near to the intimate body surface-contacting surface, at a distance of not more than 15%, preferably 10%, more preferably 5% in proportion to the total thickness of the product.

54. The tampon kit according to claim 53, characterized in that
the top sheet(s) (70b) are foldable and/or cuttable top sheets, and/or
it comprises tampons (10) of different sizes, preferably it comprises vaginal tampons (10) having different dimensions and top sheet(s) (70b) having different dimensions, which cover at least a part of the outer surface of the tampon (10) of a size, and/or
the probiotic top sheet(s) (70b) have different patterns, structures, colors and/or packaging.

55-57. (canceled)

58. The vaginal tampon according to claim 1, wherein said vaginal tampon is provided with an applicator.

59. The vaginal tampon according to claim 1, wherein the outer surface of the tampon body may be provided with a top sheet which is permeable to the metal particles.
